(19) United States
(12) Patent Application Publication
(10) Pub. No.: US 2011/0270290 A1
(43) Pub. Date: Nov. 3, 2011

(54) EAR CLEANING DEVICE AND METHOD

(76) Inventors: Yotam Nadam, Tel Aviv (IL); Igal Nadam, Tel Aviv (IL); Israel Sorian Friedman, Tel Aviv (IL)

(21) Appl. No.: 13/140,059

(22) PCT Filed: Dec. 2, 2009
(86) PCT No.: PCT/IL2009/001133

§ 371 Date: Jul. 20, 2011

(30) Foreign Application Priority Data
Dec. 21, 2008 (IL) 196083

(51) Int. Cl. A61F 11/00

(52) U.S. Cl. 606/162

(57) ABSTRACT

In one aspect the present invention is directed to an ear-cleaning device comprising: a first cotton wad, for cleaning an ear of an individual; a stick, the first cotton wad being wrapped on one end thereof, for inserting the first cotton wad into the ear; a first guarding object attached to the one end of the stick, for limiting the entrance of the stick into the ear; and detachment means of the first stick from the first guarding object, thereby preventing unintentional contact of the first cotton wad with the myringa of the ear, thereby preventing damage thereof while cleaning of the ear.

### FIELD OF THE INVENTION

The present invention relates to the field of ear cleaning. In particular, the present invention relates to the field of safe, hygienic ear cleaning.

### BACKGROUND OF THE INVENTION

The ears must regularly be cleaned of excess accumulation of wax.

Cotton tips are the most common ear-cleaning devices. The shaft of the cotton-tipped stick is either plastic or wood, manufactured to uniform length and shape.

However, the ear is a delicate, intricate area, including the skin of the ear canal and particularly the myringa (eardrum). Too vigorous attention to ear cleanliness may put hearing at risk, due to injury of the myringa.

It is an object of the present invention to provide a safe ear-cleaning device for self-use.

Other objects and advantages of the invention will become apparent as the description proceeds.

### SUMMARY OF THE INVENTION

The following embodiments and aspects thereof are described and illustrated in conjunction with systems, tools methods, and so forth, which are meant to be merely illustrative, not limiting in scope.

In one aspect the present invention is directed to an ear-cleaning device comprising:
a first cotton wad, for cleaning an ear of an individual;
a stick, the first cotton wad being wrapped on one end of the stick, for inserting the first cotton wad into the ear;
a first guarding object attached to the one end of the stick, for limiting the entrance of the stick into the ear; and
detachment means of the first stick from the first guarding object,
   thereby preventing unintentional contact of the first cotton wad with the myringa of the ear, thereby preventing damage thereof while cleaning of the ear.

The detachment means may comprise adjusting means for adjusting the distance between the first stick and the first guarding object,
thereby allowing adjustment of the limitation of the entrance into the ear.

The first guarding object may comprise an object having width greater than that of the entrance of the ear.

The ear-cleaning device may further comprise:
a second cotton wad being wrapped on the second end of the stick;
a second guarding object attached to the second end of the stick; and
detachment means of the stick from the second guarding object.

The first guarding object may comprise a thimble, for wearing the device on a finger.

The detaching means may comprise corresponding machining item between the first guarding object and the stick, the item rendering friction force between the first guarding object and the stick.

The detaching means may comprise corresponding threads of the first guarding object and the stick.

The detaching means comprise corresponding protrusions and slots between the first guarding object and the stick.

The ear-cleaning device may further comprise:
a third cotton wad; and
a third stick attached to the third cotton wad,
the third cotton wad and third stick for replacing the first stick and the first cotton wad.

The external surface of the stick may comprise a thread for wrapping cotton fibers thereon.

The external surface of the stick may comprise extensions for wrapping cotton fibers thereon.

In another aspect the present invention is directed to a method for ear-cleaning, the method comprising the steps of:
adjusting the location of a guarding object on a stick, for limiting the entrance of the stick into the ear, in order to prevent contact between the tip of the stick and the myringa of the ear; and
inserting the stick into the canal of the ear, to a depth determined by the adjustment;

thereby preventing unintentional contact of the cotton wad with the myringa of the ear, thereby preventing damage thereof while cleaning the ear.

The method may further comprise the step of wearing the guarding object on a finger.

The method may further comprise the steps of
detaching the stick from the guarding object; and
attaching another stick on the guarding object.

In addition to the exemplary aspects and embodiments described above, further aspects and embodiments will become apparent by reference to the figures and by study of the following detailed description.

### BRIEF DESCRIPTION OF THE DRAWINGS

The objects and features of the present invention will become apparent from the following detailed description considered in conjunction with the accompanying drawings, in which:

FIG. 1 illustrates a device for cleaning the ear, according to one embodiment of the present invention.

FIG. 2 illustrates the device of FIG. 1, detached.

FIG. 3 illustrates the adjustment of the length of the neck of the device of FIG. 1.

FIG. 4 is a sectional view of another embodiment for adjusting the length of neck 52.

FIG. 5 illustrates the first step of insertion of the device of FIG. 1 into the ear.

FIG. 6 illustrates the second step of insertion of the device of FIG. 1 into the ear.

FIG. 7 illustrates the third step of insertion of the device of FIG. 1 into the ear.

FIG. 8 illustrates a package for selling the device of FIG. 1.

FIG. 9 illustrates a sectional view of the structure of the cotton stick according to one embodiment.

FIG. 10 illustrates a device for cleaning the ear, according to another embodiment of the present invention.

FIG. 11 illustrates a device for cleaning the ear, according to another embodiment of the present invention.

It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, are merely intended to conceptually illustrate the structures and procedures described herein. Reference numerals may be repeated among the figures in order to indicate corresponding or analogous elements.

### DETAILED DESCRIPTION OF PREFERRED EMBODIMENTS

In the following detailed description, numerous specific details are set forth in order to provide a thorough understanding of the invention. However, it will be understood by those skilled in the art that the present invention may be practiced without these specific details. In some instances, well-known methods, procedures, components and circuits have not been described in detail, for the sake of brevity.

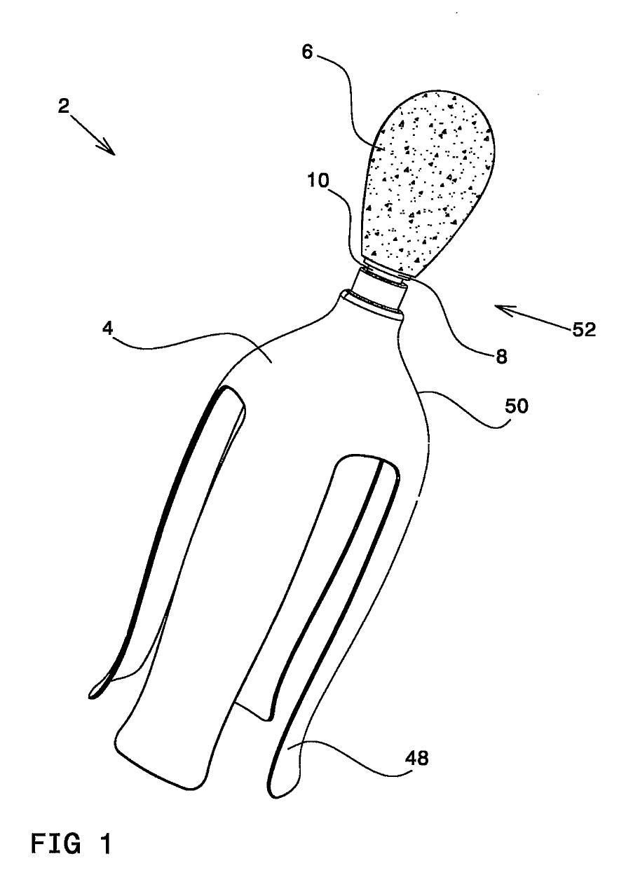

FIG. 1 illustrates a device for cleaning the ear, according to one embodiment of the present invention.

An ear-cleaning device 2 includes a thimble 4 holding an appropriate wrapped cotton wad 6 for cleaning the ear.

Thimble 4 may include springy branches 48 at the bottom for grasping an inserted finger, a narrow extension head 10 at the top, and an expansion 50 in between.

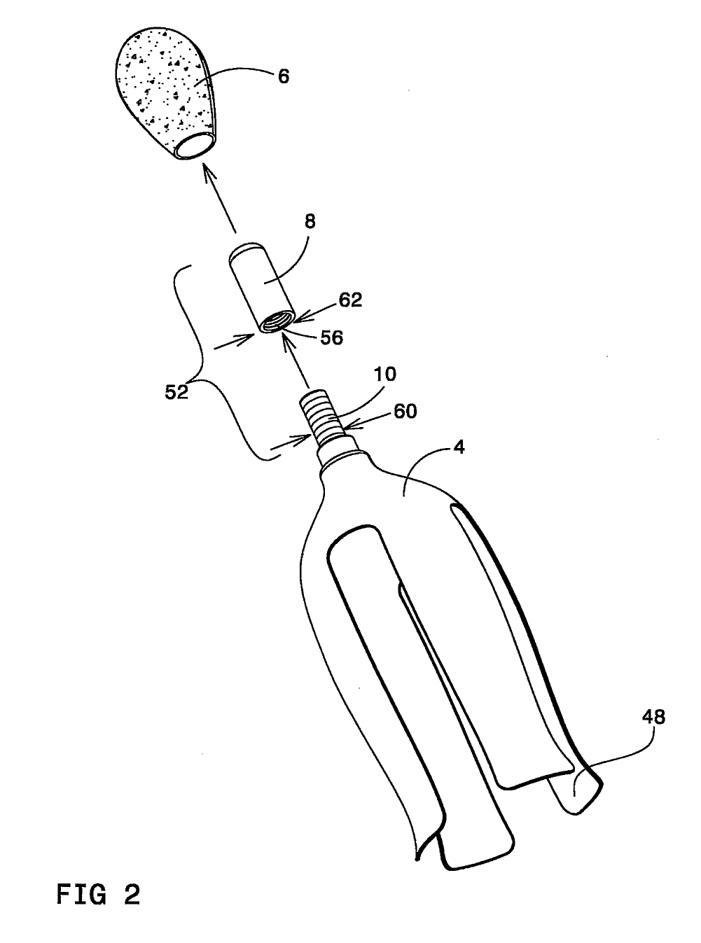

FIG. 2 illustrates the device of FIG. 1, detached.

The user may detach thimble 4 from cotton stick 8 holding cotton wad 6.

Cotton stick 8 and an extension head 10 of thimble 4 fit together, and may be adjustably extracted one from the other, to enlarge or diminish the distance between cotton wad 6 and thimble 4.

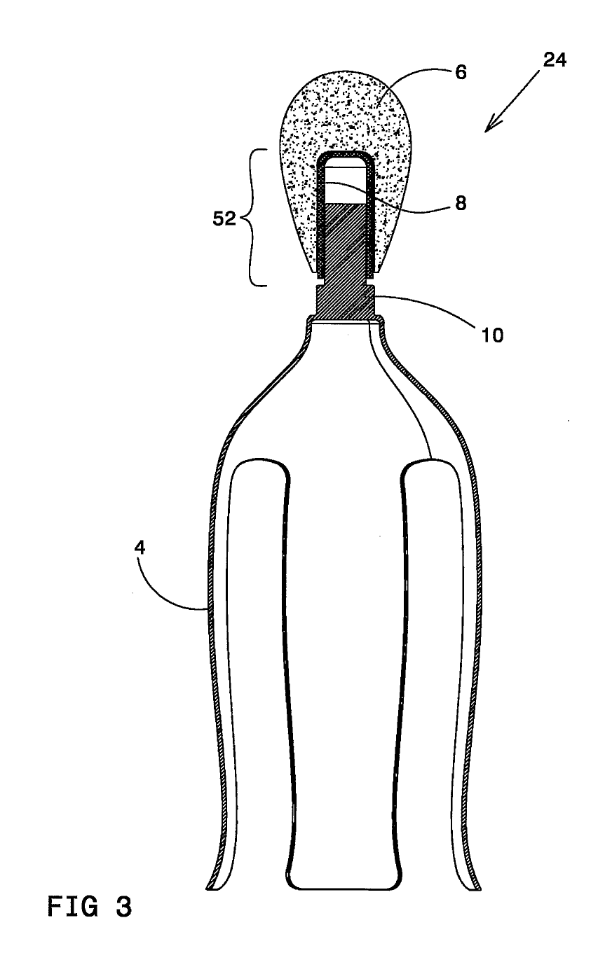

FIG. 3 illustrates the adjustment of the length of the neck of the device of FIG. 1.

The term “neck”, denoted by 52, will refer herein to the combination of cotton stick 8 and extension head 10 of thimble 4.

According to one embodiment the force holding cotton stick 8 to extension head 10, and allowing the adjustment of the length of neck 52, may be friction force between them, provided by corresponding machining item of diameter 60 of extension head 10 and diameter 62 of cotton stick 8.

This friction force should be larger than the friction force between cotton wad 6 and the ear canal walls.

According to this embodiment, the length of neck 52 may be adjusted by simple pushing or pulling of cotton stick 8 in relation to extension head 10 of thimble 4.

According to another embodiment, cotton stick 8 and extension head 10 may have corresponding threads 56 and 54 (shown in FIG. 2) for adjusting the length of neck 52.

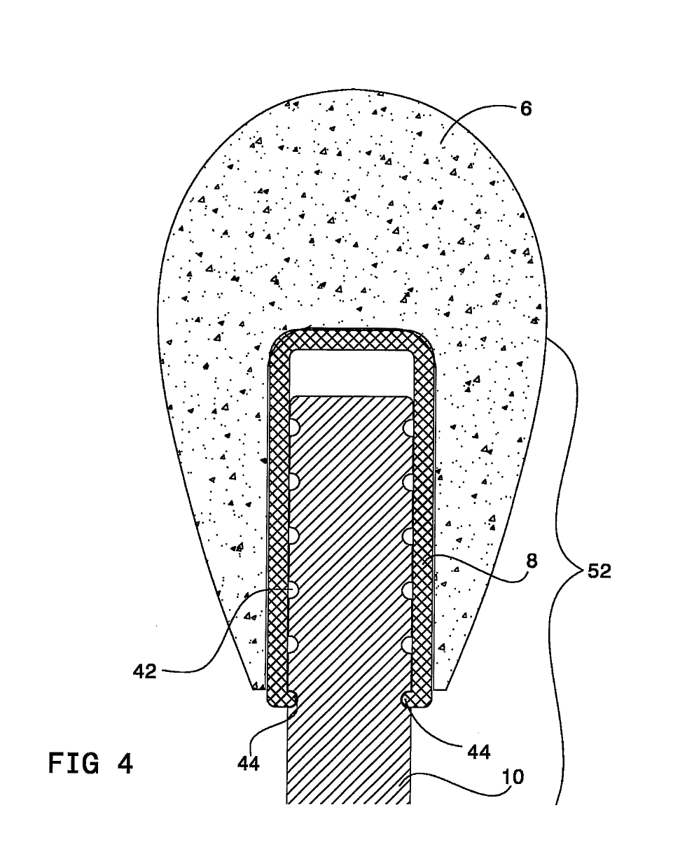

FIG. 4 is a sectional view of another embodiment for adjusting the length of neck 52.

Extension head 10 has several slots 42, each for a different length of neck 52. A protrusion 44 of cotton stick 8, corresponding to slots 42, may enter one of slots 42, to determine the desired length of neck 52.

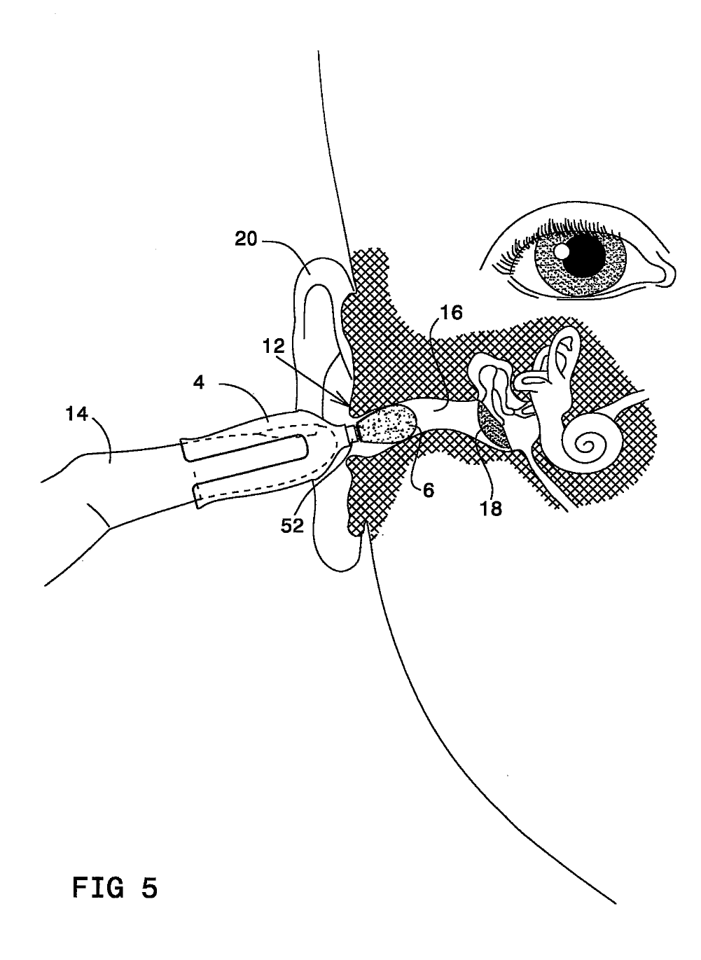

FIG. 5 illustrates the first step of insertion of the device of FIG. 1 into the ear.

Finger 14 in thimble 4 pushes cotton wad 6 through entrance 12 of the ear into canal 16 thereof.

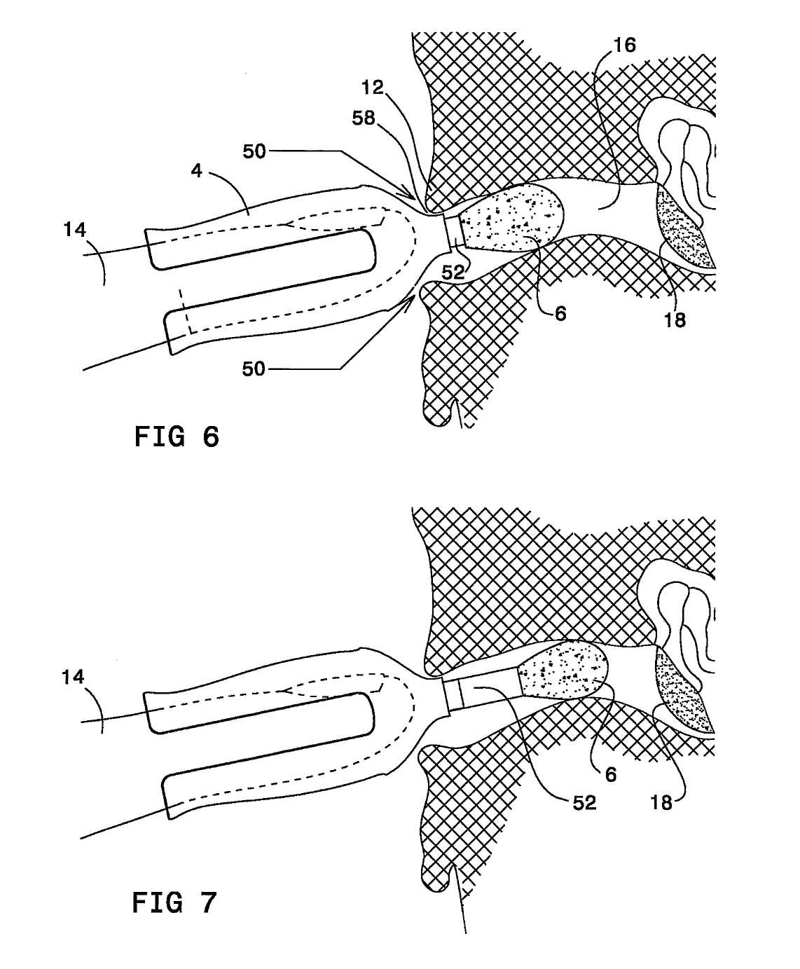

FIG. 6 illustrates the second step of insertion of the device of FIG. 1 into the ear.

The term “border” refers herein to the location on thimble 4, where thimble cannot enter ear entrance 12.

Thimble 4 includes a border 58, located on expansion 50, where the width of thimble 4 is greater than ear entrance 12.

Thimble 4 functions as a guarding object, as border 58 thereof limits insertion of cotton wad 6 into ear canal 16.

The location of border 58 is fixed and not adjustable, depending on the width of ear entrance 12.

FIG. 7 illustrates the third step of insertion of the device of FIG. 1 into the ear.

For cleaning canal 16 at greater depth, the user may extend neck 52 by extracting cotton stick 8 from extension head 10, and then reinsert cotton wad 6, subject to the limitation of fixed border 58.

The term “pre-adjustment” refers herein to the adjustment of the estimated penetration depth of cotton wad 6 into canal 16 prior to ear cleaning.

Adjustment of the length of neck 52 pre-adjusts the depth of cotton wad 6 at the actual cleaning of the ear.

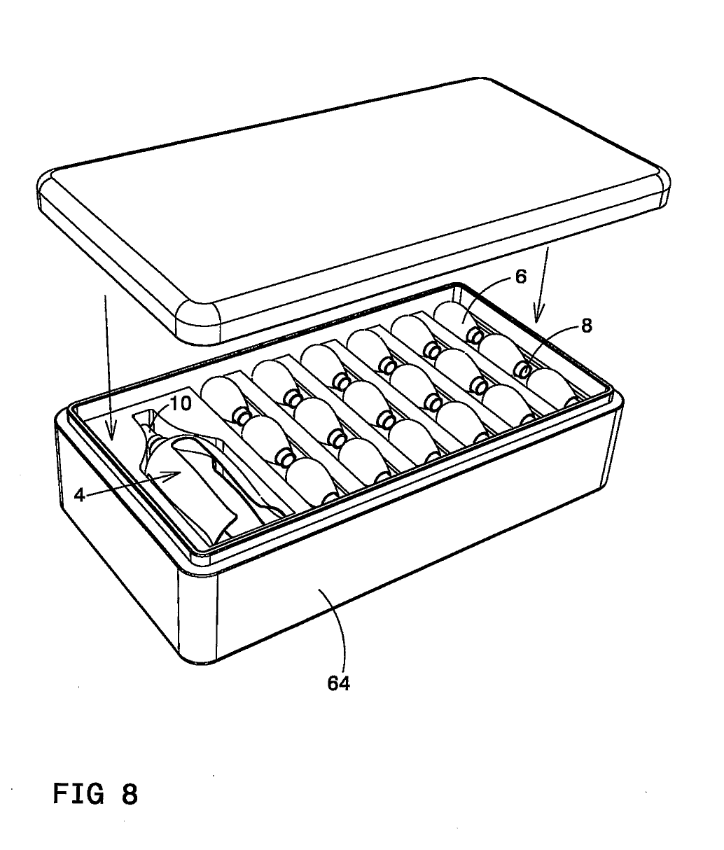

FIG. 8 illustrates a package for selling the device of FIG. 1.

The term “bulb” refers herein to a cotton stick 8 wrapped by a cotton wad 6.

Since cotton stick 8 may be attached and detached from thimble 4, a plurality of bulbs, each including cotton stick 8 and cotton wad 6, may be replaceable components sold in a package 64 with one thimble 4.

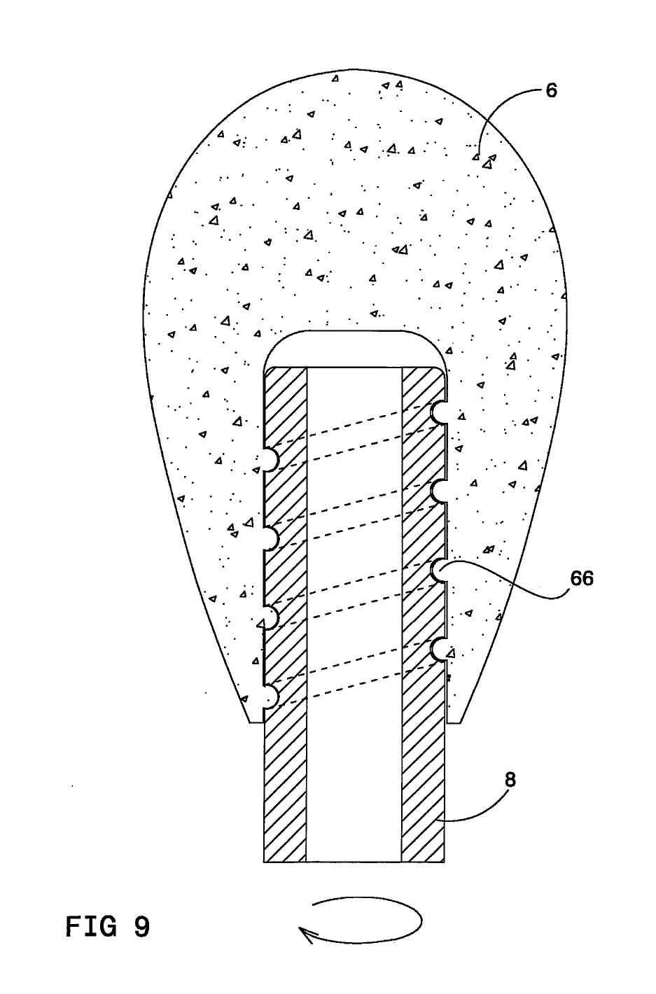

FIG. 9 illustrates a sectional view of the structure of the cotton stick according to one embodiment.

The longitudinal external surface of cotton stick 8 may include a thread 66 or niches for industrially wrapping cotton wad 6 by spinning cotton stick 8 in relation to the cotton, such that thread 66 catches the cotton fibers, producing a wad.

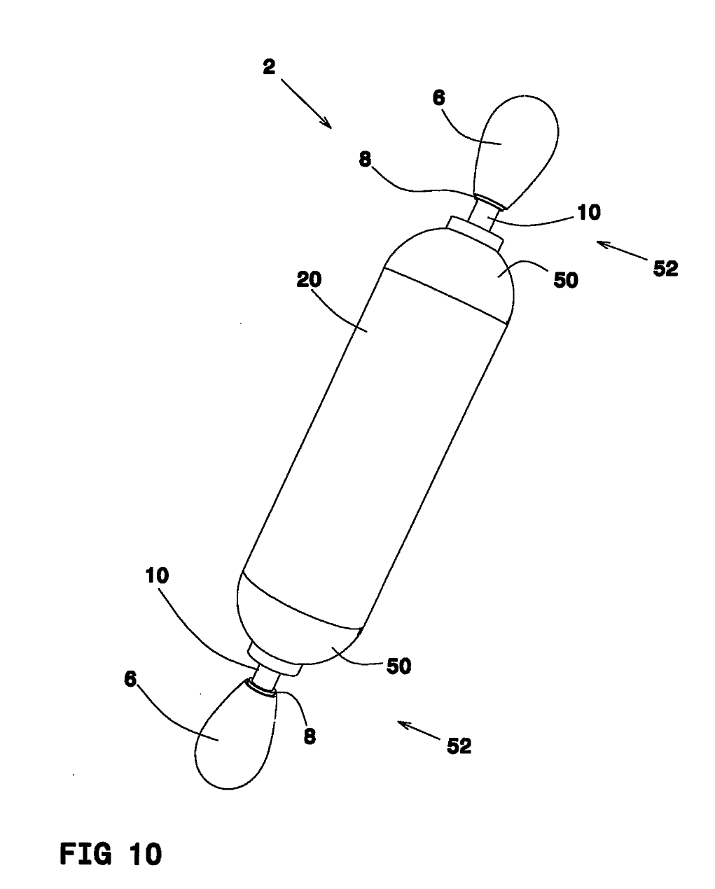

FIG. 10 illustrates a device for cleaning the ear, according to another embodiment of the present invention.

According to this embodiment ear-cleaning device 2 includes a handle 20 holding two wrapped cotton wads 6. Handle 20 includes a wide zone for grasping, two narrow extension heads 10 at the tops thereof, and expansions 50 in between. The user may detach handle 20 from each of cotton sticks 8, each holding cotton wad 6 thereof.

Each of cotton sticks 8 and extension heads 10 of handle 20 fit together, and may be adjustably extracted one from the other, to enlarge or diminish the distance between cotton wad 6 and handle 20.

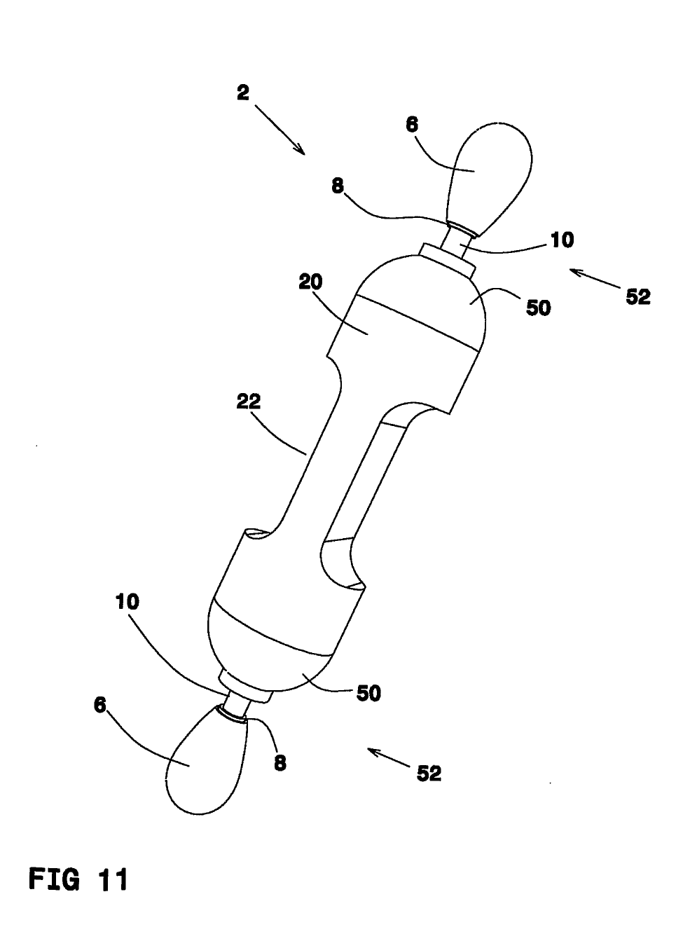

FIG. 11 illustrates a device for cleaning the ear, according to another embodiment of the present invention.

Ear-cleaning device 2 of FIG. 11 is similar to FIG. 10 except for an additional recess 22 for holding handle 20.

In the figures and description herein, the following reference numerals have been mentioned:
numeral 2 denotes an ear-cleaning device according to one embodiment of the present invention;
numeral 4 denotes a thimble of the ear-cleaning device;
numeral 6 denotes a cotton wad;
numeral 8 denotes a cotton stick for locating the cotton wad;
numeral 10 denotes an extension head, extending from the thimble;
numeral 12 denotes the entrance of the ear;
numeral 14 denotes a finger;
numeral 16 denotes an ear canal;
numeral 18 denotes a myringa (ear drum);
numeral 20 denotes a handle;
numeral 22 denotes a recess in the handle;
numeral 42 denotes a slot on the extension head for adjusting the distance between the extension head and the cotton stick;
numeral 44 denotes a protrusion of the cotton stick for adjusting the distance of the extension head from the cotton stick;
numeral 48 denotes a branch of the thimble for grasping the inserted finger;
numeral 50 denotes an expansion between the thimble head (finger width) and the extension head (narrow);
numeral 52 denotes the combination of the cotton stick and the extension head of the thimble; this combination noted as the “neck”;
numerals 54 and 56 denote corresponding threads of the cotton stick and the extension head, for adjusting the length of the neck;
numeral 58 denotes the nearest location of the thimble to the ear, where the thimble is wider than the ear entrance, said location delineated as “border”;
numeral 60 denotes the diameter of the extension head;
numeral 62 denotes the diameter of the cotton stick;
numeral 64 denotes packaging for marketing the ear cleaning device; and
numeral 66 denotes a thread on the cotton stick for industrially wrapping the cotton wad.

While certain features of the invention have been illustrated and described herein, the invention can be embodied in other forms, ways, modifications, substitutions, changes, equivalents, and so forth. The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. An ear-cleaning device comprising:
a first cotton wad, for cleaning an ear of an individual;
a stick, said first cotton wad being wrapped on one end of said stick, for inserting said first cotton wad into said ear;
a first guarding object attached to said one end of said stick, for limiting the entrance of said stick into said ear; and
detachment means of said first stick from said first guarding object,
thereby preventing unintentional contact of said first cotton wad with the myringa of said ear, thereby preventing damage thereof while cleaning of said ear.

2. The ear-cleaning device of claim 1, wherein said detachment means comprise adjusting means for adjusting the distance between said first stick and said first guarding object,
thereby allowing adjustment of said limitation of said entrance into said ear.

3. The ear-cleaning device of claim 1, wherein said first guarding object comprises an object having width greater than that of the entrance of said ear.

4. The ear-cleaning device of claim 1, further comprising:
a second cotton wad being wrapped on the second end of said stick;
a second guarding object attached to said second end of said stick; and
detachment means of said stick from said second guarding object.

5. The ear-cleaning device of claim 3, wherein said first guarding object comprises a thimble, for wearing said device on a finger.

6. The ear-cleaning device of claim 1, wherein said detaching means comprise corresponding machining item between said first guarding object and said stick, said item rendering friction force between said first guarding object and said stick.

7. The ear-cleaning device of claim 1, wherein said detaching means comprise corresponding threads of said first guarding object and said stick.

8. The ear-cleaning device of claim 1, wherein said detaching means comprise corresponding protrusions and slots between said first guarding object and said stick.

9. The ear-cleaning device of claim 1, further comprising:
a third cotton wad; and
a third stick attached to said third cotton wad, said third cotton wad and third stick for replacing said first stick and said first cotton wad.

10. The ear-cleaning device of claim 1, wherein the external surface of said stick comprises a thread for wrapping cotton fibers thereon.

11. The ear-cleaning device of claim 1, wherein the external surface of said stick comprises extensions for wrapping cotton fibers thereon.

12. A method for ear-cleaning, the method comprising the steps of:
adjusting the location of a guarding object on a stick, wrapped with cotton wad, for limiting the entrance of said stick into said ear, in order to prevent contact between the tip of said stick and the myringa of said ear; and
inserting said stick into the canal of said ear, to a depth determined by said adjustment;
thereby preventing unintentional contact of said cotton wad with the myringa of said ear, thereby preventing damage thereof while cleaning said ear.

13. The method of claim 12, further comprising the step of wearing said guarding object on a finger.

14. The method of claim 14, further comprising the steps of
detaching said stick from said guarding object; and
attaching another stick on said guarding object.
